(19) United States
(12) Patent Application Publication
(10) Pub. No.: US 2026/0076654 A1
(43) Pub. Date: Mar. 19, 2026

(54) ULTRASONIC MAGING METHOD AND SYSTEM

(71) Applicant: WUHAN UNITED IMAGING HEALTHCARE CO., LTD., Wuhan (CN)

(72) Inventors: Aiwei Shi, Wuhan (CN); Tong Li, Hubei (CN); Hao Ling, Wuhan (CN); Zhixin Tian, Wuhan (CN)

(21) Appl. No.: 19/329,526

(22) Filed: Sep. 15, 2025

(30) Foreign Application Priority Data
Sep. 14, 2024 (CN) 202411296196.3

(51) Int. Cl. A61B 8/00; A61B 8/14

(52) U.S. Cl. CPC A61B 8/5253; A61B 8/14; A61B 8/4488; A61B 8/463; A61B 8/5207

(57) ABSTRACT

Embodiments of the present disclosure may provide an ultrasonic imaging method. The method may include: for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets; obtaining at least two initial ultrasonic images based on the at least two ultrasonic sub-images for the each of the at least one ultrasonic transmission; and determining a target ultrasonic image of the scanning region by fusing the at least two initial ultrasonic images.

### CROSS-REFERENCE TO RELATED APPLICATIONS

This application claims priority to Chinese Patent Application No. 202411296196.3, filed on Sep. 14, 2024, the entire contents of which are hereby incorporated by reference.

### TECHNICAL FIELD

The present disclosure relates to the field of medical ultrasonic imaging technology, and in particular, to ultrasonic imaging methods, systems, and computer devices.

### BACKGROUND

Due to the physical characteristics of ultrasound, noise (such as speckle noise) exists in medical ultrasonic imaging. To improve ultrasonic image quality, it is desirable to provide ultrasonic imaging methods and systems that can reduce noise in ultrasonic images.

### SUMMARY

An aspect of the present disclosure may provide an ultrasonic imaging method. The method may include: for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets; obtaining at least two initial ultrasonic images based on the at least two ultrasonic sub-images for the each of the at least one ultrasonic transmission; and determining a target ultrasonic image of the scanning region by fusing the at least two initial ultrasonic images.

In some embodiments, the obtaining at least two ultrasonic sub-images of a scanning region acquired by at least two receiving array element subsets for each of at least one ultrasonic transmission may include: or the each of at least one ultrasonic transmission; determining the at least two receiving array element subsets based on a preset selection rule; acquiring ultrasonic echo signals through the at least two receiving array element subsets; and obtaining the at least two ultrasonic sub-images based on the ultrasonic echo signals.

In some embodiments, at least two ultrasonic transmissions is performed and the obtaining the at least two initial ultrasonic images based on the ultrasonic sub-images may include: for each of the at least two ultrasonic transmissions, obtaining serial numbers of the at least two receiving array element subsets, and obtaining the at least two initial ultrasonic images by coherently compounding at least two ultrasonic sub-images obtained based on ultrasonic echo signals collected by receiving array element subsets with the same serial number for the at least two ultrasonic transmissions.

In some embodiments, the receiving array element subsets with the same serial number may include different array element sets on an ultrasonic probe; or the receiving array element subsets with the same serial number may have different receiving apertures on the ultrasonic probe.

In some embodiments, the determining a target ultrasonic image of the scanning region by fusing the at least two initial ultrasonic images may include: obtaining at least two processed initial ultrasonic images by performing envelope detection on each of the at least two initial ultrasonic images; and obtaining the target ultrasonic image of the scanning region by performing incoherent compounding on the at least two processed initial ultrasonic images.

In some embodiments, for one of the at least one ultrasonic transmission, each of the at least two receiving array element subsets may include different elements on an ultrasonic probe; or each of the at least two receiving array element subsets may have different receiving apertures on the ultrasonic probe.

In some embodiments, the at least two receiving array element subsets comprise a first receiving array element subset and a second receiving array element subset; the first receiving array element subset and the second receiving array element subset include the same elements on an ultrasonic probe, or the first receiving array element subset and the second receiving array element subset have the same receive aperture on the ultrasonic probe; and elements of the first receiving array element subset and elements of the second receiving array element subset have different receive apodization schemes.

In some embodiments, at least two ultrasonic transmissions are performed. A count of the at least two receiving array element subsets for each of the at least two ultrasonic transmissions is the same.

In some embodiments, a count of the at least two receiving array element subsets is two. Receiving apertures of the at least two receiving array element subsets are symmetrical relative to a transmission center of an ultrasonic probe.

In some embodiments, a count of the at least two receiving array element subsets is two. Receiving apertures of the at least two receiving array element subsets are asymmetrical relative to a transmission center of an ultrasonic probe.

In some embodiments, each of the at least two receiving array element subsets includes all elements on an ultrasonic probe or include a portion of the all elements on the ultrasonic probe.

According to some aspects of the present disclosure, an ultrasonic imaging system may be provided. The ultrasonic imaging system may include: a storage device, a processor, and instructions, being stored in the storage device, when executed by the processor, causing the ultrasonic imaging system to perform operations including: for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets; obtaining at least two initial ultrasonic images based on the at least two ultrasonic sub-images for the each of the at least one ultrasonic transmission; and determining a target ultrasonic image of the scanning region by fusing the at least two initial ultrasonic images.

In some embodiments, the ultrasonic imaging system may further include a display, and the processor further causes the ultrasonic imaging system to perform operations including: displaying the target ultrasonic image.

In some embodiments, the obtaining at least two ultrasonic sub-images of a scanning region acquired by at least two receiving array element subsets for each of at least one ultrasonic transmission may include: or the each of at least one ultrasonic transmission; determining the at least two receiving array element subsets based on a preset selection rule; acquiring ultrasonic echo signals through the at least two receiving array element subsets; and obtaining the at least two ultrasonic sub-images based on the ultrasonic echo signals.

In some embodiments, at least two ultrasonic transmissions is performed and the obtaining the at least two initial ultrasonic images based on the ultrasonic sub-images may include: for each of the at least two ultrasonic transmissions, obtaining serial numbers of the at least two receiving array element subsets, and obtaining the at least two initial ultrasonic images by coherently compounding at least two ultrasonic sub-images obtained based on ultrasonic echo signals collected by receiving array element subsets with the same serial number for the at least two ultrasonic transmissions.

In some embodiments, the receiving array element subsets with the same serial number may include different array element sets on an ultrasonic probe; or the receiving array element subsets with the same serial number may have different receiving apertures on the ultrasonic probe.

In some embodiments, the determining a target ultrasonic image of the scanning region by fusing the at least two initial ultrasonic images may include: obtaining at least two processed initial ultrasonic images by performing envelope detection on each of the at least two initial ultrasonic images; and obtaining the target ultrasonic image of the scanning region by performing incoherent compounding on the at least two processed initial ultrasonic images.

In some embodiments, for one of the at least one ultrasonic transmission, each of the at least two receiving array element subsets may include different elements on an ultrasonic probe; or each of the at least two receiving array element subsets may have different receiving apertures on the ultrasonic probe.

In some embodiments, the at least two receiving array element subsets comprise a first receiving array element subset and a second receiving array element subset; the first receiving array element subset and the second receiving array element subset include the same elements on an ultrasonic probe, or the first receiving array element subset and the second receiving array element subset have the same receive aperture on the ultrasonic probe; and elements of the first receiving array element subset and elements of the second receiving array element subset have different receive apodization schemes.

In some embodiments, at least two ultrasonic transmissions are performed. A count of the at least two receiving array element subsets for each of the at least two ultrasonic transmissions is the same.

In some embodiments, a count of the at least two receiving array element subsets is two. Receiving apertures of the at least two receiving array element subsets are symmetrical relative to a transmission center of an ultrasonic probe.

In some embodiments, a count of the at least two receiving array element subsets is two. Receiving apertures of the at least two receiving array element subsets are asymmetrical relative to a transmission center of an ultrasonic probe.

In some embodiments, each of the at least two receiving array element subsets includes all elements on an ultrasonic probe or include a portion of the all elements on the ultrasonic probe.

According to some aspects of the present disclosure, a non-transitory computer readable medium may be provided. The non-transitory computer readable medium may include at least one set of instructions, wherein when executed by one or more processors of a computing device, the at least one set of instructions causes the computing device to perform an ultrasonic imaging method comprising: for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets; obtaining at least two initial ultrasonic images based on the at least two ultrasonic sub-images for the each of the at least one ultrasonic transmission; and determining a target ultrasonic image of the scanning region by fusing the at least two initial ultrasonic images.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures, and wherein:

FIG. 1 is a flowchart illustrating an exemplary process for an ultrasonic imaging method;

FIG. 2 provides a schematic diagram illustrating an exemplary ultrasonic imaging process;

FIG. 3 is a schematic diagram illustrating an exemplary process for multiple ultrasonic sub-images formed by a single ultrasonic transmission;

FIG. 4 is a schematic diagram illustrating an exemplary process for a coherent compounding image formed by multiple ultrasonic sub-images;

FIG. 5 is a schematic diagram illustrating an exemplary process for generating a target ultrasonic image;

FIG. 6 is a schematic diagram illustrating exemplary elements of a 1.5D ultrasonic probe;

FIG. 7 is a schematic diagram of at least a portion of receiving apertures of a 1D ultrasonic probe;

FIG. 8 is a schematic diagram illustrating an example of symmetrically allocated receiving apertures;

FIG. 9 is a schematic diagram illustrating an example of asymmetrically allocated receiving apertures;

FIG. 10 is a schematic diagram illustrating exemplary receiving apertures;

FIG. 11 is a flowchart illustrating an exemplary process for an ultrasonic imaging method;

FIG. 12 is a schematic diagram illustrating an exemplary ultrasonic imaging device; and

FIG. 13 is a schematic diagram illustrating an exemplary computer device.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections or assemblies of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The terms “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

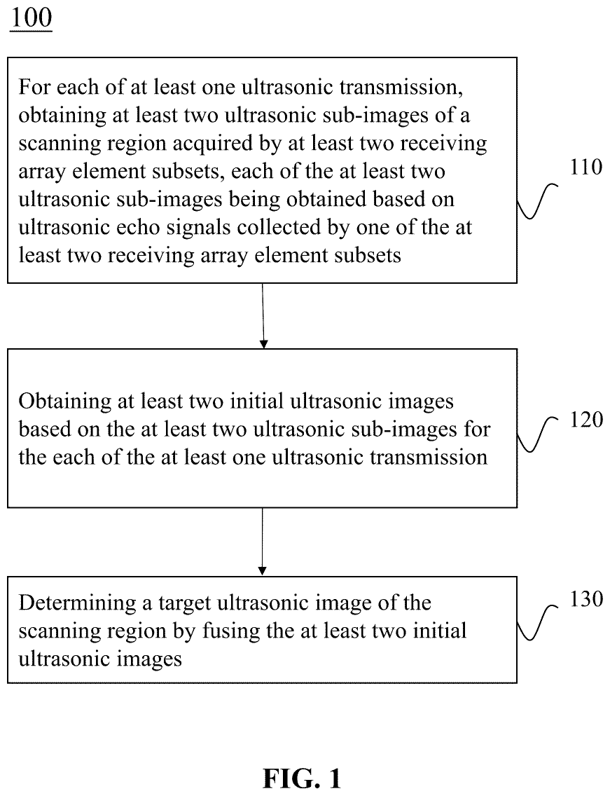

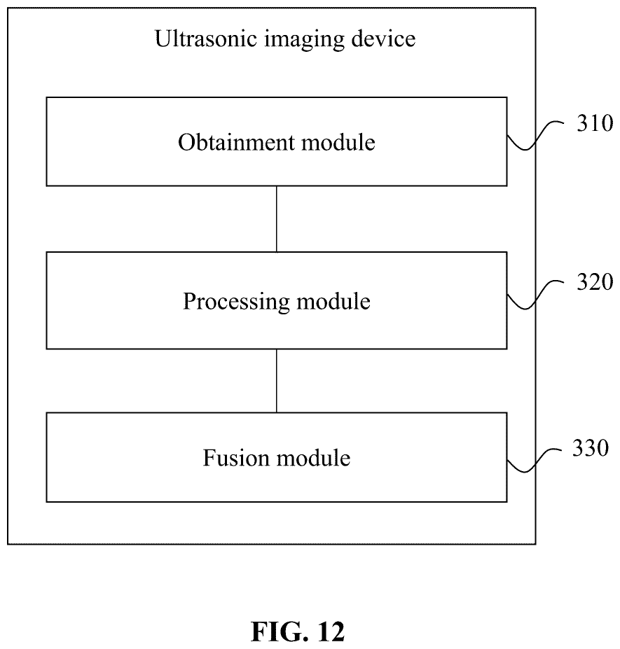

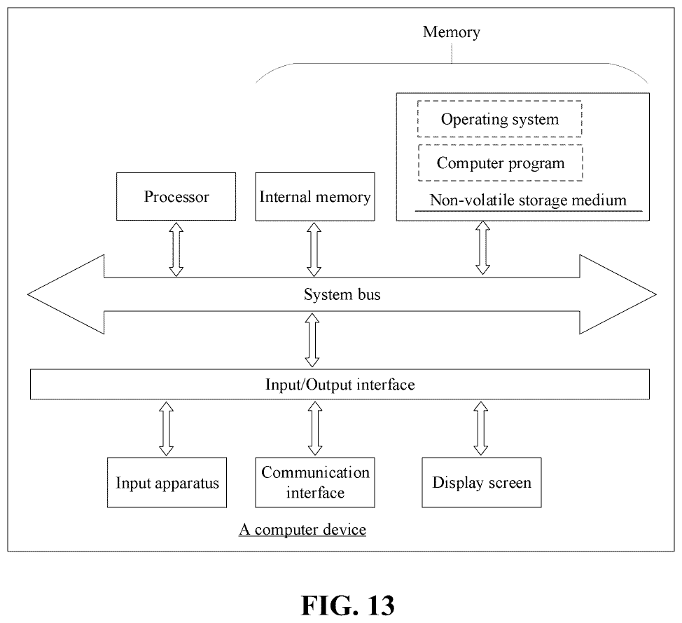

FIG. 1 is a flowchart illustrating an exemplary process for an ultrasonic imaging method. In some embodiments, process 100 may be performed by a processor (e.g., a processor in FIG. 13) or an ultrasonic imaging device (e.g., ultrasonic imaging device in FIG. 12). For example, the process 100 may be stored in the form of instructions in a storage device, and the process 100 may be implemented when the processor or the ultrasonic imaging device executes the instructions.

In 110, for each of at least one ultrasonic transmission, at least two ultrasonic sub-images of a scanning region acquired by at least two receiving array element subsets may be obtained. In some embodiments, each of the at least two ultrasonic sub-images may be obtained based on ultrasonic echo signals collected by one of the at least two receiving array element subsets.

In some embodiments, each of the at least two receiving array element subsets may include a group of receiving array elements (also referred to as a set of receiving array elements) (also referred to as elements) (also referred to as receiving array elements) (also referred to as array elements) (e.g., transducer elements) of an ultrasonic probe. The group of receiving array elements may be used to receive ultrasonic echoes, and convert the received ultrasonic echoes into electrical signals. For example, the ultrasonic probe may include a plurality of transducer elements, and a group of receiving array elements may be a portion of the plurality of transducer elements or be all of the plurality of transducer elements. In some embodiments, each of the at least two receiving array element subsets may have a receiving aperture. The receiving aperture may be formed by a group of receiving array elements selected to receive ultrasonic echoes, and to convert the received ultrasonic echoes into electrical signals. Once the receiving aperture is determined, the group of receiving array elements may be determined. In some embodiments, a group of receiving array elements on the ultrasonic probe and a receiving aperture may have an one-to-one correspondence. The electrical signals may be processed by receiving channels. A receiving array element and a a receiving channel may have a one-to-one correspondence, or multiple receiving array elements may correspond to a receiving channel.

In some embodiments, the scanning region may be an area requiring ultrasonic imaging. Each of the at least two ultrasonic sub-images may be determined based on ultrasonic echo signals collected by one of the at least two receiving array element subsets for one ultrasonic transmission.

In some embodiments, for one ultrasonic transmission, two of the at least two receiving array element subsets may be different. If two receiving array element subsets include different elements, the two receiving array element subsets may be deemed to be different. If two receiving array element subsets have different receiving apertures, the two receiving array element subsets may be deemed to be different.

In some embodiments, for each ultrasonic transmission, the at least two receiving array element subsets may be numbered, respectively. The at least two receiving array element subsets may have different series numbers. In some embodiments, receiving array element subsets with the same series number for different ultrasonic transmissions may be the same or different. In some embodiments, a count of the at least two receiving array element subsets in different ultrasonic transmissions may be the same or different.

In some embodiments, the receiving array element subsets may be determined by presetting a selection rule. During a scanning process, for the each of at least one ultrasonic transmission, the at least two receiving array element subsets may be determined based on the preset selection rule. The ultrasonic probe may acquire ultrasonic echo signals from the scanning region through the at least two receiving array element subsets, and transmit the ultrasonic echo signals to a computer device (e.g., the processor, the ultrasonic imaging device). The computer device may perform beamforming on ultrasonic echo signals collected by a receiving array element subset to obtain one ultrasonic sub-image of the scanning region. In some embodiments, the preset selection rule may be default settings. In some embodiments, the preset selection rule may be determined based on a network model (e.g., a deep learning model). The network model may be trained based on scanning conditions used by users during multiple ultrasonic scans of the scanning region and ultrasonic image quality. In some embodiments, the scanning conditions may include receiving apertures, transmission parameters, etc.

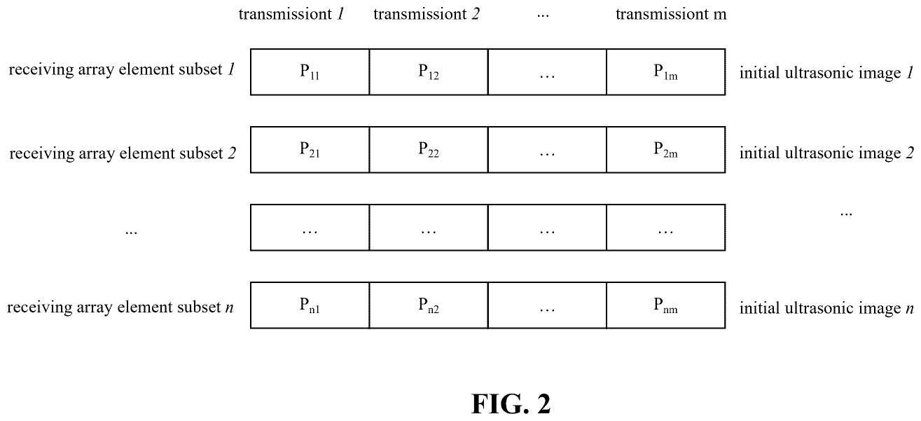

FIG. 2 provides a schematic diagram illustrating an exemplary ultrasonic imaging process. As shown in FIG. 2, during the scanning process, for the first ultrasonic transmission, n receiving array element subsets may be determined based on the preset selection rule, n ultrasonic sub-images of the scanning region may be acquired through the n receiving array element subsets, such as P₁₁, P₁₂ . . . , Pₙ₁. One of the n ultrasonic sub-image may correspond to one subset of the n receiving array element subsets, that is, the one ultrasonic sub-image may be obtained based on ultrasonic signals collected by the one receiving array element subset. Similarly, for the second ultrasonic transmission, another n receiving array element subsets may be determined based on the preset selection rule, and another n ultrasonic sub-images of the scanning region may be acquired through the another n receiving array element subsets, such as P₁₂, P₂₂, . . . , Pₙ₂. This process may continue iteratively. If the scanning process involves m ultrasonic transmissions, that is, a count of the at least one ultrasonic transmission is m, the scanning process may terminate until another n ultrasonic sub-images of the scanning region are acquired through another n receiving array element subsets for the m^{th} ultrasonic transmission (i.e., the last ultrasonic transmission), such as P₁ₘ, P₂ₘ, . . . , Pₙₘ.

FIG. 2 is merely an illustrative example of the ultrasonic imaging process, and does not show overlapping between ultrasonic sub-images. In some embodiments, for one ultrasonic transmission, ultrasonic echo signals received by two receiving array element subsets may be from the same location of the scanning region, and there may be overlapping between the ultrasonic sub-images obtained based on the two receiving array element subsets, resulting in imaging of the same location.

In some embodiments, for different ultrasonic transmissions, the receiving array element subsets (e.g., determined based on the preset selection rule) may be different. In some embodiments, for each ultrasonic transmission, the at least two receiving array element subsets may be numbered. At least one receiving array element subset with the same series number in the at least one ultrasonic transmission may be different. For example, receiving array element subset 1 of the first ultrasonic transmission and receiving array element subset 1 of the second ultrasonic transmission may include different sets of array elements on the ultrasonic probe, or have different receiving apertures on the ultrasonic probe. In some embodiments, the count of the at least two receiving array element subsets for two different ultrasonic transmissions may be the same or different.

In 120, at least two initial ultrasonic images may be obtained based on the at least two ultrasonic sub-images for the each of the at least one ultrasonic transmission.

In some embodiments, the receiving array element subset(s) with the same series number corresponding to each ultrasonic transmission are set to be the same. Each of the at least two initial ultrasonic images may be obtained based on at least one ultrasonic sub-image acquired by the same receiving array element subset for the at least one ultrasonic transmission. In some embodiments, if a count of the at least one ultrasonic transmission is 1, only one ultrasonic sub-image may be obtained for each of the at least two receiving array elements, and the only ultrasonic sub-image may be directly determined as the initial ultrasonic image for each of the at least two receiving array elements. In some embodiments, if a count of the at least one ultrasonic transmission is two or more, two or more ultrasonic sub-images is obtained for each of the at least two receiving array elements, coherent compounding may be performed on the two or more ultrasonic sub-images to obtain the initial ultrasonic image for the each of the at least two receiving array elements.

As shown in FIG. 2, if the count of the at least one ultrasonic transmission is 1, the obtained ultrasonic sub-images (P₁₁, P₁₂, . . . , Pₙ₁) may each be determined as one initial ultrasonic image. If there are m (m≥2) ultrasonic transmissions, for receiving array element subset 1, m ultrasonic sub-images (P₁₁, P₁₂ . . . , P₁ₘ) may be obtained. Coherent compounding may be performed on the m ultrasonic sub-images (P₁₁, P₁₂, . . . , P₁ₘ) to obtain the first initial ultrasonic image for the receiving array element subset 1. For receiving array element subset 2, coherent compounding may be performed on m ultrasonic sub-images (P₂₁, P₂₂, . . . , P₂ₘ) to obtain the second initial ultrasonic image, and so on, until for receiving array element subset n, coherent compounding may be performed on m ultrasonic sub-images (Pₙ₁, Pₙ₂, . . . , Pₙₘ) to obtain the n^{th} initial ultrasonic image.

In 130, a target ultrasonic image of the scanning region may be determined by fusing the at least two initial ultrasonic images.

In some embodiments, the target ultrasonic image may be used for diagnosis and/or treatment.

In some embodiments, the at least two initial ultrasonic images corresponding to the at least two receiving array element subsets may be fused. Specifically, envelope detection may be performed on the at least two initial ultrasonic images, and then incoherent compounding may be performed on the at least two initial ultrasonic images after the envelope detection to obtain the target ultrasonic image of the scanning region.

In some embodiments, as shown in FIG. 2, envelope detection may be performed on the first initial ultrasonic image, the second initial ultrasonic image, . . . , and the n^{th} initial ultrasonic image, respectively, and then incoherent compounding may be performed on the first initial ultrasonic image, the second initial ultrasonic image, . . . , and the n^{th} initial ultrasonic image after the envelope detection to obtain the target ultrasonic image.

The above ultrasonic imaging method may include: acquiring ultrasonic sub-images of the scanning region from the at least two receiving array element subsets, stitching ultrasonic sub-images corresponding to each of the at least two receiving array element subsets (with the same series number) to obtain the initial ultrasonic images, and then fusing the initial ultrasonic images to produce the target ultrasonic image of the scanning region. During the scanning process, multiple initial ultrasonic images of the scanning region can be obtained through multiple receiving array element subsets. Since each initial image exhibits different speckle characteristics, the target ultrasonic image determined based on the fused result may have reduced speckle noise. Moreover, the ultrasonic sub-images used to generate the multiple initial images can be simultaneously acquired during each ultrasonic transmission in the scanning process. Even if the scanning region moves, a high-quality target ultrasonic image can still be obtained without degradation in resolution or reduction of the occurrence of image trailing artifacts.

In some embodiments, operation 110 may include: for the each of the at least one ultrasonic transmission, determining the at least two receiving array element subsets based on the preset selection rule; acquiring ultrasonic echo signals through the at least two receiving array element subsets; and obtaining the at least two ultrasonic sub-images based on the ultrasonic echo signals.

In some embodiments, the preset selection rule may be default settings or automatically determined by a neural network model. The default settings may be pre-configured based on experience, e.g., before the device leaves the factory.

In some embodiments, for each ultrasonic transmission, the computer device may determine the at least two receiving array element subsets for the ultrasonic probe according to the preset selection rule. The computing device may instruct the ultrasonic probe to collect ultrasonic echo signals through the at least two receiving array element subsets. The ultrasonic probe simultaneously may obtain at least two sets of ultrasonic echo signals, and then transmit the at least two sets of ultrasonic echo signals to the computer device. The computing device may convert each set of the ultrasonic echo signals into an ultrasonic sub-image, resulting in the at least two ultrasonic sub-images.

In some embodiments, taking a one dimensional (1D) ultrasonic probe as an example, the count of the at least two receiving array element subsets may be 3. The preset selection rule for determining the three receiving array element subsets is set as follows. The distribution center of receiving array element subset 1 may be aligned with the transmission center of the ultrasonic probe (also referred to as ultrasonic probe transmission center). The distribution center of receiving array element subset 2 may be offset to the left of the ultrasonic probe transmission center. The distribution center of receiving array element subset 3 may be offset to the right of the ultrasonic probe transmission center. As shown in FIG. 2, for the first ultrasonic transmission, after determining the three receiving array element subsets of the ultrasonic probe according to the preset selection rule, the ultrasonic sub-image Pu may be obtained from the ultrasonic echo signals collected by receiving array element subset 1, the ultrasonic sub-image P₂₁ may be obtained from the ultrasonic echo signals collected by receiving array element subset 2, and the ultrasonic sub-image P₃₁ may be obtained from the ultrasonic echo signals collected by receiving array element subset 3, respectively. For the second ultrasonic transmission, three receiving array element subsets determined according to the preset selection rule may differ from those of the first ultrasonic transmission, ultrasonic sub-images (P₁₂, P₂₂, P₃₂) may be obtained. This process may continue iteratively until ultrasonic sub-images (P₁ₘ, P₂ₘ, P₃ₘ) may be obtained for the m^{th} ultrasonic transmission.

In some embodiments, for each ultrasonic transmission, the at least two receiving array element subsets may be determined according to the preset selection rule. By collecting ultrasonic echo signals through the at least two receiving array element subsets, the at least two ultrasonic sub-images corresponding to each ultrasonic transmission may be obtained. Thus multiple ultrasonic sub-images of the scanning region may be acquired simultaneously during a single ultrasonic transmission, avoiding degradation in imaging quality caused by movement (e.g., physical movement, physiological movement) of the scanning region.

In some embodiments, a count of the at least one ultrasonic transmission may be at least two, that is, at least two ultrasonic transmissions may be performed. Operation 120 may include: for each of the at least two ultrasonic transmissions, obtaining serial numbers of the at least two receiving array element subsets, and obtaining the at least two initial ultrasonic images by coherently compounding at least two ultrasonic sub-images with the same serial number for the at least two ultrasonic transmissions, respectively. In some embodiments, a count of the at least two initial ultrasonic images may be equal to a count of the at least two receiving array element subsets set for each of the at least two ultrasonic transmissions.

In some embodiments, for each ultrasonic transmission, the at least two receiving array element subsets may be numbered manually or automatically. The serial numbers (also referred to as series numbers) may be assigned to each receiving array element subset and may be set based on the preset selection rule. For example, the serial numbers may be default setting or automatically set by a network model. The default settings may be pre-configured based on experience, e.g., before the device leaves the factory. The network model may be determined by training a preliminary network model using training samples. The network may be configured to output the serial numbers of the receiving array element subsets. Coherent compounding may be used to superimpose the ultrasonic sub-images.

In some embodiments, after determining the at least two receiving array element subsets according to the preset selection rule, the computer device may assign a serial number (also referred to a series number) to each receiving array element subset. Additionally, after at least two ultrasonic sub-images are obtained for each ultrasonic transmission, the computer device may number the at least two ultrasonic sub-images based on the serial numbers of the receiving array element subsets. For example, a receiving array element subset and an ultrasonic sub-image acquired by the receiving array element subset may have the same series number. When the count of the at least one ultrasonic transmissions is two or more, the computer device may, after the scanning process is completed, obtain serial numbers of the at least two receiving array element subsets corresponding to the ultrasonic sub-images, and perform coherent compounding on two or more ultrasonic sub-images acquired by receiving array element subsets with the same serial number to obtain one initial ultrasonic image.

In some embodiments, as shown in FIG. 2, the at least two receiving array element subsets determined according to the preset selection rule may be sequentially numbered as: 1,2,3,4,5 . . . n. For the first ultrasonic transmission, an ultrasonic sub-image with serial number 1 obtained from receiving array element subset 1 is denoted as Pu, an ultrasonic sub-image with serial number 2 obtained from receiving array element subset 2 is denoted as P₂₁, . . . , an ultrasonic sub-image with serial number n obtained from receiving array element subset n is denoted as Pₙ₁, and so on, until the m^{th} ultrasonic transmission (m≥2), an ultrasonic sub-image with serial number 1 obtained from receiving array element subset 1 is denoted as P₁ₘ, an ultrasonic sub-image with serial number 2 obtained from receiving array element subset 2 is denoted as P₂ₘ, . . . , an ultrasonic sub-image with serial number n obtained from receiving array element subset n is denoted as Pₙₘ. After the scanning process is completed, the ultrasonic sub-images corresponding to the receiving array element subset 1 may be obtained, and initial ultrasonic image 1 may be obtained by performing coherent compounding on ultrasonic sub-images corresponding to the receiving array element subset 1. Then, the ultrasonic sub-images corresponding to the receiving array element 2 are obtained, and initial ultrasonic image 2 may be obtained by performing coherent compounding on ultrasonic sub-images corresponding to the receiving array element subset 2. This process may continue until the ultrasonic sub-images corresponding to the receiving array element n are obtained, and initial ultrasonic image n may be obtained by performing coherent compounding on ultrasonic sub-images corresponding to the receiving array element subset n.

If the count of the at least one ultrasonic transmission is one, that is, there is only one ultrasonic transmission, the computer device may directly use each ultrasonic sub-image as the initial ultrasonic image, thereby obtaining the initial ultrasonic image corresponding to each receiving array element subset. As shown in FIG. 2, the ultrasonic sub-image Pu may be directly used as the initial ultrasonic image 1, the ultrasonic sub-image P₂₁ may be directly used as the initial ultrasonic image 2, . . . , and the ultrasonic sub-image Pₙ₁ may be directly used as the n^{th} initial ultrasonic image.

As described above, for the at least two ultrasonic transmissions, by acquiring the series numbers of the receiving array element subsets corresponding to the ultrasonic sub-images, and performing coherent compounding on the at least two ultrasonic sub-images with the same series number to obtain the at least two initial ultrasonic images, multiple ultrasonic sub-images obtained during the scanning process may be coherently compounded into ultrasonic images with higher signal-to-noise ratios, thereby improving the quality of ultrasonic imaging.

In some embodiments, among the at least two ultrasonic transmissions, at least two receiving array element subsets with the same series number may include different sets of array elements on the ultrasonic probe, or have different receiving apertures on the ultrasonic probe.

In some embodiments, the computer device may assign serial numbers to the at least two receiving array element subsets determined according to the preset selection rule in each ultrasonic transmission. When there are two or more ultrasonic transmissions, there may be at least two receiving array element subsets with the same series number. The at least two receiving array element subsets with the same series number may include different sets of array elements on the ultrasonic probe, or have different receiving apertures on the ultrasonic probe.

In some embodiments, receiving array element subsets with the same series number for different ultrasonic transmissions may be different. For illustration purpose, in the first ultrasonic transmission, receiving array element subset 7, receiving array element subset 2, and receiving array element subset 3 may be determined according to the preset selection rule. In the second ultrasonic transmission, receiving array element subset 1, receiving array element subset 2, and receiving array element subset 3 may also be determined according to the preset selection rule. The receiving array element subset 2 in the first ultrasonic transmission and the receiving array element subset 2 in the second ultrasonic transmission may have different sets of array elements on the ultrasonic probe, or have different receiving apertures on the ultrasonic probe. Taking a 1D ultrasonic probe as an example, the distribution center of receiving array element subset 2 in the first ultrasonic transmission may have a receiving aperture with a radius of 3 elements and be offset to the left of the ultrasonic probe transmission center, while the distribution center of receiving array element subset 2 in the second ultrasonic transmission may have a receiving aperture with a radius of 4 elements and be offset to the left of the ultrasonic probe transmission center.

As described above, in the at least two ultrasonic transmissions, the at least two receiving array element subsets with the same serial number may have different sets of array element on the ultrasonic probe, or have different receiving apertures on the ultrasonic probe. For each of the at least two ultrasonic transmissions, the receiving array element subsets determined by the preset selection rule may be associated with the same serial number, thereby ensuring coherent imaging and improving imaging quality.

In some embodiments, operation 130 may include: obtaining at least two processed initial ultrasonic images by performing envelope detection on each of the at least two initial ultrasonic images; and obtaining the target ultrasonic image of the scanning region by performing incoherent compounding on the at least two processed initial ultrasonic images.

Incoherent compounding may be a processing method for superimposing images after envelope detection.

In some embodiments, the envelope detection may be performed on each initial ultrasonic image to obtain the at least two processed initial ultrasonic images. Subsequently, incoherent compounding may be performed on all of the at least two processed ultrasonic images to derive the target ultrasonic image of the scanning region.

In some embodiments, as shown in FIG. 2, after coherently compounding the ultrasonic sub-images into the at least two initial ultrasonic images, n initial ultrasonic images may be obtained. Envelope detection may be performed on each initial ultrasonic image to obtain n processed initial ultrasonic images. Incoherent compounding of the n processed ultrasonic images may produce the target ultrasonic image.

As described above, by performing envelope detection on each initial ultrasonic image to obtain the at least two processed initial ultrasonic images, and then performing incoherent compounding on the at least two processed initial ultrasonic images, the target ultrasonic image of the scanning region may be obtained. Therefore, multiple initial ultrasonic images with different speckle characteristics may be fused to reduce speckle noise, thereby improving the quality of ultrasonic imaging.

In some embodiments, each of the at least two receiving array element subsets may include different element sets on the ultrasonic probe. In some embodiments, each of the at least two receiving array element subsets may have different receiving aperture on the ultrasonic probe.

In some embodiments, for one ultrasonic transmission, different receiving array element subsets may have different sets of array elements on the ultrasonic probe, or have different receiving apertures on the ultrasonic probe.

Taking a 1.5 dimensional (1.5D) ultrasonic probe as an example, receiving array element subset 1 may include elements on the upper side and the lower side of the ultrasonic probe, receiving array element subset 2 may include elements of the middle row on the ultrasonic probe, and receiving array element subset 3 may include all elements of the ultrasonic probe. Taking a 1D ultrasonic probe as another example, receiving array element subset 1 may have a receiving aperture 1 whose distribution center is aligned with the ultrasonic probe transmission center, receiving array element subset 2 may have receiving aperture 2 whose distribution center is offset to the left of the ultrasonic probe transmission center, and receiving array element subset 3 may have receiving aperture 3 whose distribution center is offset to the right of the ultrasonic probe transmission center.

As described above, for each ultrasonic transmission, each receiving array element subset may include different element sets on the ultrasonic probe, or have different receiving apertures on the ultrasonic probe, multiple different receiving array element subsets can be formed. During the scanning process, multiple initial ultrasonic images may be obtained by using the multiple different receiving array element subsets and the target ultrasonic image may be determined by processing the multiple initial ultrasonic images, thereby reducing speckle noise in the target ultrasonic image.

In some embodiments, if two receiving array element subsets include different elements, the two receiving array element subsets may be deemed to be different. If two receiving array element subsets have different receiving apertures, the two receiving array element subsets may be deemed to be different.

In some embodiments, the at least two receiving array element subsets may include a first receiving array element subset and a second receiving array element subset. The first receiving array element subset and the second receiving array element subset are defined as two different receiving array element subsets. In some embodiments, when the first receiving array element subset and the second receiving array element subset include the same receiving array elements on the ultrasonic probe, or have the same receiving aperture on the ultrasonic probe, the first receiving array element subset and the second receiving array element subset may include different receive apodizations. For example, different receive apodizations may be assigned to ultrasonic signals collected by the first receiving array element subset and the second receiving array element subset, respectively, and ultrasonic sub-images may be determined based on the ultrasonic signals with the different receive apodizations. For illustration purposes, the first receiving array element subset and the second receiving array element subset may be two of the at least two receiving array element subsets.

In some embodiments, during one ultrasonic transmission, the first receiving array element subset and the second receiving array element subset are defined as two different receiving array element subsets, and include the same elements on the ultrasonic probe or have the same receiving aperture on the ultrasonic probe, then different receive apodizations may be configured for the first receiving array element subset and the second receiving array element subset for generating the ultrasonic sub-images, thereby ensuring that the first receiving array element subset and the second receiving array element receive ultrasonic echo signals through different receive apodizations.

As described above, when the first receiving array element subset and the second receiving array element subset include the same elements on the ultrasonic probe or have the same receiving aperture on the ultrasonic probe, different receive apodizations are applied to the first receiving array element subset and the second receiving array element subset. Thus multiple ultrasonic images may be obtained by altering the receiving apodizations, ensuring the reduction of speckle noise in the target ultrasonic image.

Some embodiments of the present disclosure may provide a method to improve the quality of ultrasonic imaging. First, for each ultrasonic transmission, a portion or all of the receiving array elements may be selected to form multiple receiving array element subsets. Each receiving array element set may include a portion of the receiving array elements or all of the receiving array elements. A count of the multiple receiving array element subsets may be greater than or equal to 2. Coherent beamforming may be performed on ultrasonic echo signals collected by each receiving array element subset, thereby generating multiple ultrasonic sub-images for each transmission. Next, ultrasonic sub-images corresponding to the first receiving array element subset from all transmissions may be selected for coherent compounding to form the first coherently compounded image (e.g., the first initial ultrasonic image described above). Similarly, ultrasonic sub-images corresponding to the second receiving array element subset from all transmissions may be selected for coherent compounding to form the second coherently compounded image (e.g., the second initial ultrasonic image described above), and so on, resulting in multiple coherently compounded images. Finally, the multiple coherently compounded images may be incoherently compounded to form a high-quality ultrasonic image (e.g., the target ultrasonic image described above). In some embodiments, only when two array element subsets include the same array elements or have the same aperture, different receive apodization schemes may be assigned during coherent beamforming, that is, for different array element subsets, there is no need to assign receive apodization schemes or the same receive apodization scheme may be assigned. In some embodiments, each receiving array element subset may be set with different receive apodization schemes during coherent beamforming. As used herein, receive apodization refers to applying different amplitude weights to signals collected by each receiving array element in the ultrasonic probe to optimize beam characteristics. In some embodiments, the same receive apodization scheme may be set by applying a fixed weighting function (e.g., Hamming window, Hanning window, etc.) to all receive array elements. In some embodiments, different receive apodization schemes may be set by: dynamically adjusting a weighting function based on detection depth or target characteristics. The different receive apodization schemes may include segmented dynamic apodization, adaptive apodization, etc. The segmented dynamic apodization may include switching the optimal apodization function for different depth intervals (e.g., a Gaussian window for near-field, a rectangular window for far-field). The adaptive apodization may include: real-time calculation of channel weights (e.g., minimum variance algorithm) based on echo signal statistical properties (e.g., coherence, noise level). In some embodiments, when two receive array element subsets include different elements or have different receiving apertures, same or different receive apodization schemes may be adopted, or there is no need to assign receive apodization schemes. When the two receive element subsets include the same elements or have the same receiving aperture, different receive apodization schemes may be used.

In some embodiments, the methods for improving the quality of ultrasonic imaging mentioned above may be illustrated below.

In S1, all receiving array element subsets corresponding to each ultrasonic transmission may be determined, thereby generating different receiving array element subsets for each ultrasonic transmission.

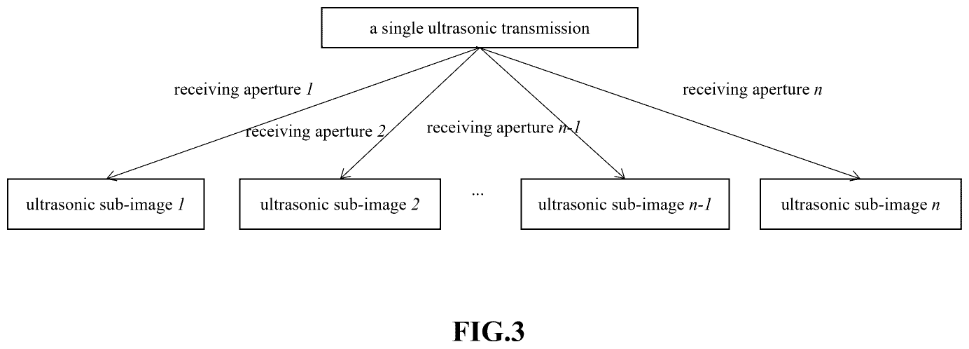

In S2, coherent compounding may be performed on signal collected by the different receiving array element subsets to obtain different ultrasonic sub-images. FIG. 3 is a schematic diagram illustrating an exemplary process for multiple ultrasonic sub-images formed by a single ultrasonic transmission, the different receiving array element subsets may have receiving aperture 1, receiving aperture 2, . . . , receiving aperture n−1, receiving aperture n, and the obtained different ultrasonic sub-images may be ultrasonic sub-image 1, ultrasonic sub-image 2, . . . , ultrasonic sub-image n−1, ultrasonic sub-image n.

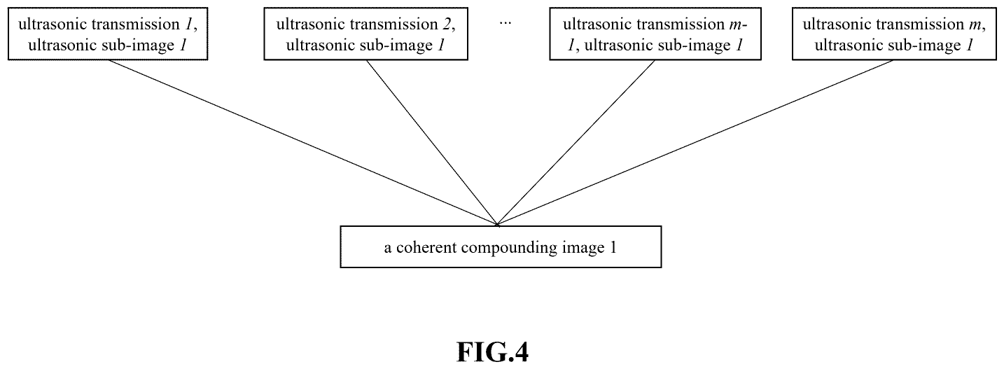

In S3, for each receiving array element subset (with the same series number), coherent compounding may be performed on all ultrasonic sub-images acquired from each ultrasonic transmission to obtain a full ultrasonic image (e.g., the initial ultrasonic image as described above) (e.g., the coherent compounding image). FIG. 4 is a schematic diagram illustrating an exemplary process for a coherent compounding image formed by multiple ultrasonic sub-images. For receiving aperture 1, ultrasonic sub-images acquired from each transmission may be ultrasonic sub-image 1 for ultrasonic transmission 1, ultrasonic sub-image 1 for ultrasonic transmission 2, . . . , ultrasonic sub-image 1 for (m−1)^{th} ultrasonic transmission, ultrasonic sub-image 1 for m^{th} h ultrasonic transmission. The m ultrasonic sub-images obtained from the m ultrasonic transmissions may be coherently compounded to generate coherent compounding image 1 (e.g., the initial ultrasonic image as described above) corresponding to receiving aperture 1. Coherent compounding image 1 may be a full ultrasonic image. n receiving array element subsets may be set for each ultrasonic transmission, resulting in n coherent compounding ultrasonic images.

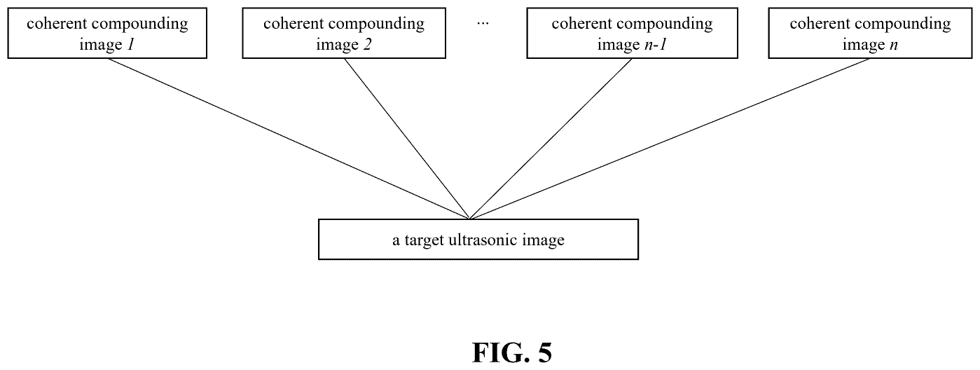

In S4, envelope detection may be performed on the coherent compounding ultrasonic image corresponding to each receiving array element subset, then incoherent compounding may be performed on all envelope-detected ultrasonic images corresponding to the receiving array element subsets to obtain a target ultrasonic image, as the result of spatial compounding, as shown in FIG. 5.

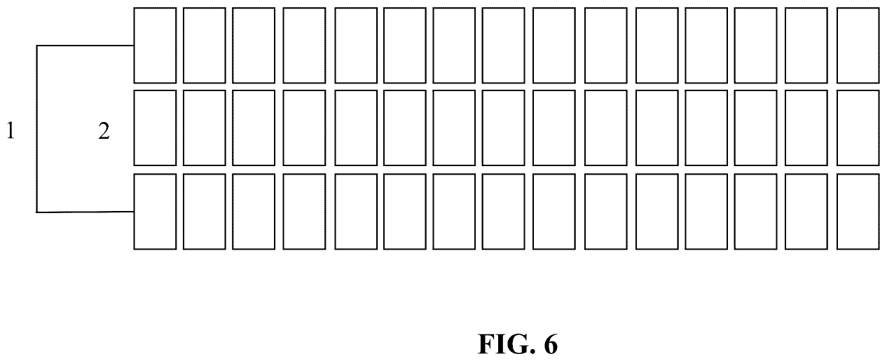

In some embodiments, the above method may be applied to ultrasonic imaging with a 1.5D ultrasonic probe. FIG. 6 is a schematic diagram illustrating exemplary elements of a 1.5D ultrasonic probe. Referring to FIG. 6, the 1.5D ultrasonic probe may include three rows of elements. Elements in the first row and the third row may be connected in pairs. Each block refers to an element. In FIG. 6, “1” represents signals obtained from elements in the first row and the third row. “2” represents signals obtained from elements in the second row. The elements of the first row and the third row may be assigned as receiving aperture 1, the elements of the second row may be assigned as receiving aperture 2, and all elements of the three rows of elements may be assigned as receiving aperture 3. Coherent compound imaging is performed on the ultrasonic sub-images collected from receive aperture 1, receive aperture 2, and receive aperture 3, respectively, to obtain coherent compounding ultrasonic image 1, coherent compounding ultrasonic image 2, and coherent compounding ultrasonic image 3. After envelope detection of the three coherent compounding ultrasonic images, the target ultrasonic image is determined through incoherent compounding.

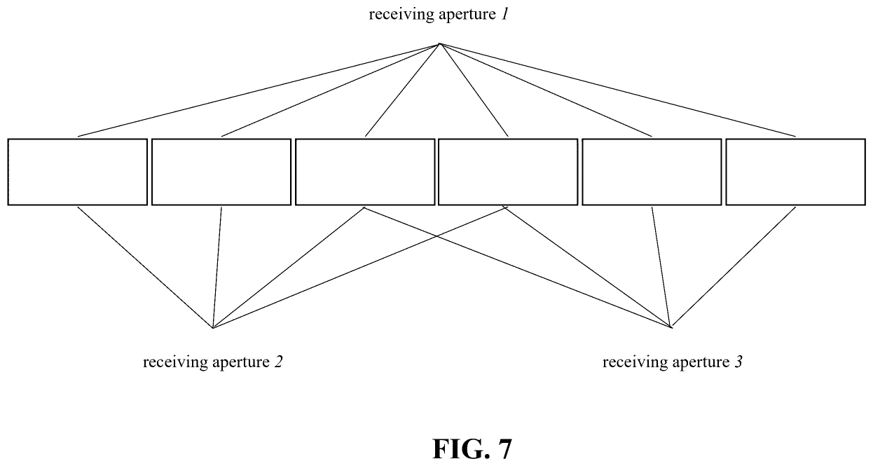

In some embodiments, the above method may also be applied to ultrasonic imaging with a 1D ultrasonic probe. FIG. 7 is a schematic diagram of at least a portion of receiving apertures of a 1D ultrasonic probe. Referring to FIG. 7, receiving apertures for each transmission may be divided into three parts, resulting in three receiving apertures. The distribution center of the first receiving aperture (i.e., receiving aperture 1 in FIG. 7) may align with the transmission center of the ultrasonic probe. The distribution center of the second receiving aperture (i.e., receiving aperture 2 in FIG. 7) may be offset to the left relative to the transmission center of the ultrasonic probe. The distribution center of the third receiving aperture (i.e., receiving aperture 3 in FIG. 7) is offset to the right relative to the transmission center of the ultrasonic probe.

In some embodiments, the distribution center of a receiving aperture may be determined based on an array element set corresponding to the receiving aperture. The transmission center of the ultrasonic probe may be determined based on a transmitting array element set. In one embodiment, the count of array elements corresponding to the union of the at least two receiving array element subsets for an ultrasonic transmission is P. The first receiving aperture corresponds to a set of array elements including array element 1, array element 2, array element Q, and array element L. The distribution center of the first receiving aperture is at: 1/P*(1+2+3+Qₚ+Lₚ), where Qₚ and Lₚ represent positions of the corresponding receiving array elements. Referring to receiving aperture 1 in FIG. 7, P is 6, receiving aperture 1 corresponds to 6 array elements, and the distribution center of receiving aperture 1 is at 3.5 array elements. The center of each array element may be defined as the position of the array element. The distribution center of receiving aperture 1 is located between the third array element and fourth array element (e.g., from left to right). The calculation method for the transmission center is the same as that for the distribution center of the receiving aperture. An ultrasonic sub-image may be obtained corresponding to receiving aperture 1 for each ultrasonic transmission. All ultrasonic sub-images formed by all receiving apertures 1 for all transmissions are coherently compounded to obtain coherently compounding image 1. Similarly, all ultrasonic sub-images formed by all receiving apertures 2 for all transmissions are coherently compounded to obtain coherently compounding image 2, and all ultrasonic sub-images formed by all receiving apertures 3 for all transmissions are coherently compounded to obtain coherently compounding image 3. FIG. 5 is a schematic diagram illustrating an exemplary process for generating a target ultrasonic image, after envelope detection of the three coherently compounding images, the target ultrasonic image may be formed through incoherent compounding.

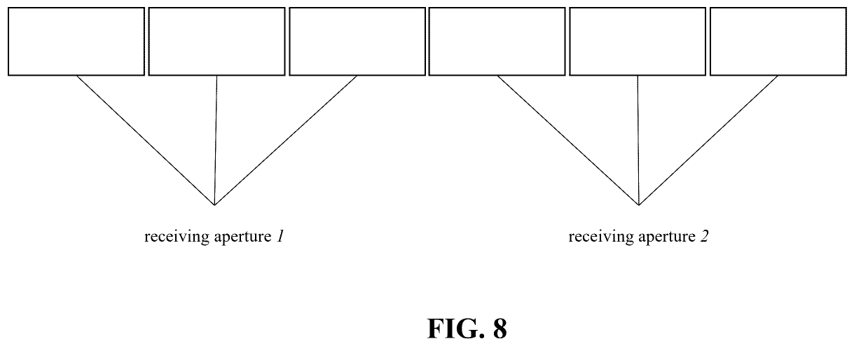

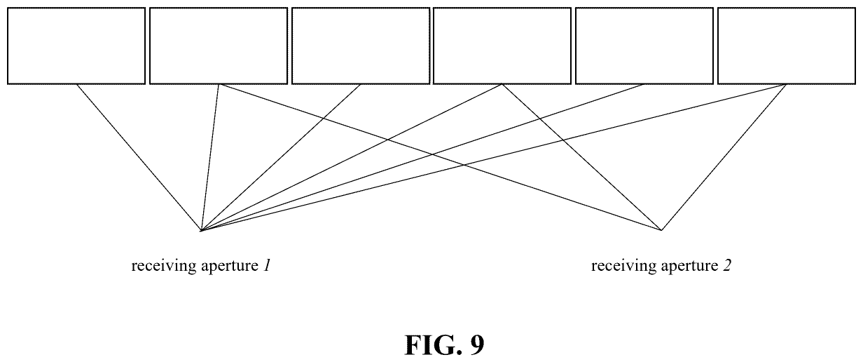

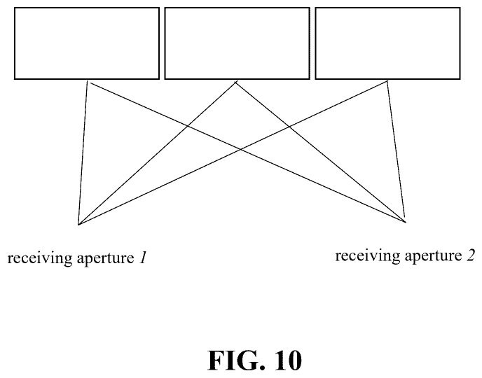

It should be noted that the count of the receiving array element subsets or the receiving apertures is not limited to 3, and the centers of the receiving apertures may not all be located on the left (or right) side of the ultrasonic probe transmission center. For example, when the count of the receiving apertures is two, that is, the count of the receiving array element subsets is two, receiving aperture 1 and receiving aperture 2 may be symmetrically allocated relative to the ultrasonic probe transmission center as shown in FIG. 8. Alternatively, receiving aperture 1 and receiving aperture 2 may be asymmetrically allocated relative to the ultrasonic probe transmission center as shown in FIG. 9. In some embodiments, when receiving aperture 1 and receiving aperture 2 are the same, different receive apodization schemes may be applied to receiving aperture 1 and receiving aperture 2, as specifically illustrated in FIG. 10.

The method for improving ultrasonic imaging quality allows multiple ultrasonic images to be formed from a single ultrasonic emission, and each of the multiple ultrasonic images may have distinct speckle characteristics. By performing compounding after envelope detection, the speckle noise in the ultrasonic images can be reduced, resulting in smoother and more uniform ultrasonic images. Moreover, traditional spatial compounding schemes require emitting ultrasonic waves at different angles to generate multiple ultrasonic images, and tissue motion can easily lead to reduced resolution and introduce motion artifacts. By generating multiple ultrasonic images for a single ultrasonic emission in the present disclosure, the potential hazards of resolution degradation and image artifacts may be alleviated. Furthermore, since the ultrasonic images are derived from coherent compounding of multiple emissions, higher resolution and signal-to-noise ratio can be maintained in the ultrasonic images.

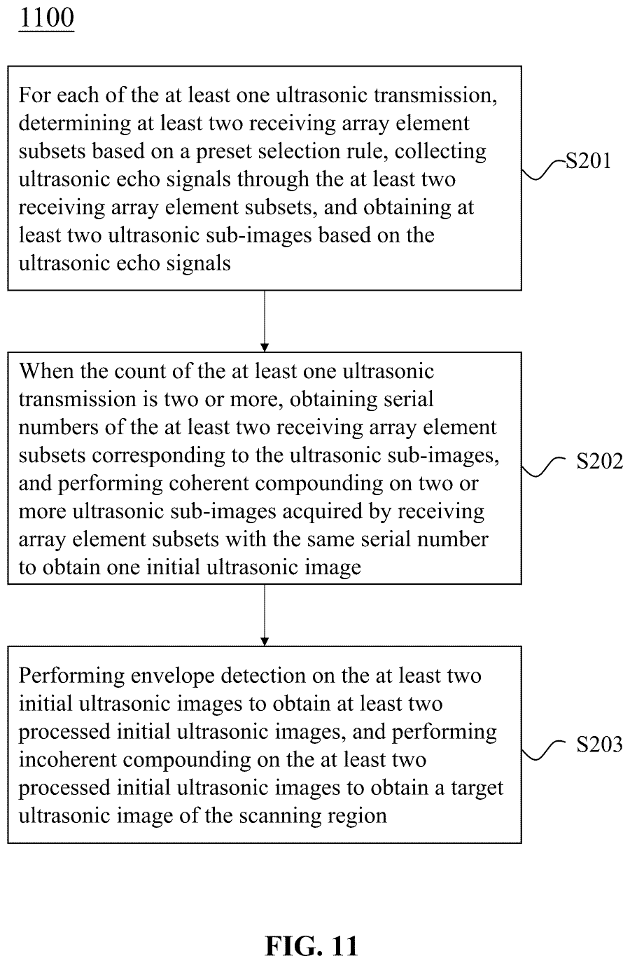

FIG. 11 is a flowchart illustrating an exemplary process for an ultrasonic imaging method. In some embodiments, process 1100 may be performed by a processor (e.g., a processor in FIG. 13) or an ultrasonic imaging device (e.g., ultrasonic imaging device in FIG. 12). For example, the process 1100 may be stored in the form of instructions in a storage device, and the process 1100 may be implemented when the processor or the ultrasonic imaging device executes the instructions.

In 201, for each of the at least one ultrasonic transmission, at least two receiving array element subsets may be determined based on a preset selection rule. Ultrasonic echo signals may be collected through the at least two receiving array element subsets, and at least two ultrasonic sub-images may be obtained based on the ultrasonic echo signals. Each of the at least two ultrasonic sub-images may be obtained based on ultrasonic echo signals collected by one of the at least two receiving array element subsets.

In 202, when the count of the at least one ultrasonic transmission is two or more, after the scanning process is completed, serial numbers of the at least two receiving array element subsets corresponding to the ultrasonic sub-images may be obtained, and coherent compounding may be performed on two or more ultrasonic sub-images acquired by receiving array element subsets with the same serial number to obtain one initial ultrasonic image, thereby obtaining at least two initial ultrasonic images

In 203, envelope detection may be performed on the at least two initial ultrasonic images to obtain at least two processed initial ultrasonic images, then incoherent compounding may be performed on the at least two processed initial ultrasonic images to obtain a target ultrasonic image of the scanning region.

In some embodiments, for the each of at least one ultrasonic transmission, two or more receiving array element subsets may be determined based on the preset selection rule. The ultrasonic probe may acquire ultrasonic echo signals from the scanning region through the two or more receiving array element subsets, and transmit the ultrasonic echo signals to a computer device. Then the computing device may determine two or more ultrasonic sub-images based on the received ultrasonic echo signals. If a count of the at least one ultrasonic transmission is 1, the computer device may determine each ultrasonic sub-image obtained by each of the at least two receiving array elements as an initial ultrasonic image, resulting in two or more initial ultrasonic images corresponding to the two or more receiving array element subsets. When a count of the at least one ultrasonic transmissions is two or more, the computer device may obtain serial numbers of the receiving array element subsets, and may perform coherent compounding on the two or more ultrasonic sub-images acquired by receiving array element subsets with the same serial number to obtain one initial ultrasonic image. The computer device may perform envelope detection on each of the at least two initial ultrasonic images to obtain the at least two processed initial ultrasonic images, and obtain the target ultrasonic image of the scanning region by performing incoherent compounding on the at least two processed initial ultrasonic images.

In some embodiments, for each of the at least one ultrasonic transmission, the at least two receiving array element subsets may be determined based on the preset selection rule. Ultrasonic echo signals may be collected through the at least two receiving array element subsets, and the at least two ultrasonic sub-images may be obtained based on the ultrasonic echo signals. When a count of the at least two ultrasonic transmission may be at least two, serial numbers of the at least two receiving array element subsets may be obtained, the at least two initial ultrasonic images may be obtained by coherently compounding at least two ultrasonic sub-images with the same serial number for the at least two ultrasonic transmissions, at least two processed initial ultrasonic images may be obtained by performing envelope detection on each of the at least two initial ultrasonic images; and the target ultrasonic image of the scanning region may be obtained by performing incoherent compounding on the at least two processed initial ultrasonic images. This approach enables the acquisition of multiple initial ultrasonic images of the scanning region through multiple receiving array element subsets during the scanning process. Since each initial ultrasonic image exhibits distinct speckle characteristics, their fusion result, i.e., the target ultrasonic image may have reduced speckle noise. Moreover, the ultrasonic sub-images used to generate the multiple initial ultrasonic images can be simultaneously acquired during each ultrasonic transmission in the scanning process. Even if the scanning region moves, a high-quality target ultrasonic image can still be obtained with less resolution degradation or image trailing artifacts. More descriptions regarding process 1100 may be found elsewhere in the present disclosure, for example, process 100 in FIG. 1.

In some embodiments, an ultrasonic imaging system may be provided. The ultrasonic imaging system may include an ultrasonic probe, a processor, and a display. For each of at least one ultrasonic transmission, the ultrasonic probe is configured to collect ultrasonic echo signals from a scanning region through at least two receiving array element subsets. The processor may be configured to obtain at least two initial ultrasonic images based on the ultrasonic echo signals, and determine a target ultrasonic image of the scanning region by fusing the at least two initial ultrasonic images. The display may be configured to display the target ultrasonic image.

In some embodiments, for each of the at least one ultrasonic transmission, the at least two receiving array element subsets may be determined based on a preset selection rule. The ultrasonic probe may acquire the ultrasonic echo signals from the scanning region through the two or more receiving array element subsets. The processor may then determine at least two ultrasonic sub-images based on the ultrasonic echo signals. If a count of the at least one ultrasonic transmission is 1, the processor may determine each ultrasonic sub-image obtained by each of the at least two receiving array elements as an initial ultrasonic image, resulting in the at least two initial ultrasonic images corresponding to the receiving array element subsets. When a count of the at least one ultrasonic transmission is two or more, the processor may obtain the serial numbers of the receiving array element subsets, perform coherent compounding on two or more ultrasonic sub-images with the same serial number to obtain one initial ultrasonic images. The processor may perform envelope detection on each of the at least two initial ultrasonic images to obtain at least two processed initial ultrasonic images, and obtain the target ultrasonic image of the scanning region by performing incoherent compounding on the at least two processed initial ultrasonic images.

The ultrasonic imaging system may acquire ultrasonic sub-images of the scanning region from the at least receiving array element subsets. Based on the ultrasonic sub-images corresponding to each of the at least receiving array element subsets, at least two initial ultrasonic images may be generated. By fusing the at least two initial ultrasonic images, the target ultrasonic image of the scanning region may be produced. During the scanning process, multiple initial ultrasonic images of the scanning region can be obtained through multiple subsets of receiving array elements. Since each initial ultrasonic image exhibits different speckle characteristics, the fused target ultrasonic image achieves reduced speckle noise. Moreover, the ultrasonic sub-images used to generate the multiple initial ultrasonic images can be simultaneously acquired during each ultrasonic emission in the scanning process. Even if the scanning region moves, a high-quality target ultrasonic image can still be obtained with less resolution degradation or image trailing artifacts. More descriptions regarding the ultrasonic imaging system may be found elsewhere in the present disclosure, for example, process 100 in FIG. 1.

FIG. 12 is a schematic diagram illustrating an exemplary ultrasonic imaging device. The ultrasonic imaging device may include an obtainment module 310, a processing module 320, and a fusion module 330.

The obtainment module 310 may be configured to obtain at least two ultrasonic sub-images of a scanning region acquired by at least receiving array element subsets for each of at least one ultrasonic transmission.

The processing module 320 may be configured to obtain at least two initial ultrasonic images based on the at least two ultrasonic sub-images for the each of the at least one ultrasonic transmission.

The fusion module 330 may be configured to determine a target ultrasonic image of the scanning region by fusing the at least two initial ultrasonic images.

In some embodiments, the obtainment module 310 may further be configured to: for the each of at least one ultrasonic emission, determine at least two receiving array element subsets based to a preset selection rule; acquire ultrasonic echo signals through the at least two receiving array element subsets; and obtain the at least two ultrasonic sub-images based on the ultrasonic echo signals.

In some embodiments, the processing module 320 may further be configured to: when the count of the at least one ultrasonic transmission is two or more, after the scanning process is completed, obtain serial numbers of the at least two receiving array element subsets corresponding to the ultrasonic sub-images, and perform coherent compounding on two or more ultrasonic sub-images acquired by receiving array element subsets with the same serial number to obtain one initial ultrasonic image.

In some embodiments, among the at least two of the ultrasonic emissions, the receiving array element subsets with the same serial number may include different array element sets on an ultrasonic probe or have different receiving apertures on the ultrasonic probe.

In some embodiments, the fusion module 330 may be further configured to: obtain at least two processed initial ultrasonic images by performing envelope detection on each of the at least two initial ultrasonic images; and obtain the target ultrasonic image of the scanning region by performing incoherent compounding on the at least two processed initial ultrasonic images.

In some embodiments, each of the at least two receiving array element subsets may have different array element sets on the ultrasonic probe, or have different receiving apertures on the ultrasonic probe.

In some embodiments, the at least two receiving array element subsets may include a first receiving array element subset and a second receiving array element subset. If the first receiving array element subset and the second receiving array element subset may include the same receiving array elements on the ultrasonic probe, or have the same receiving aperture on the ultrasonic probe, the first receiving array element subset and the second receiving array element subset may include different receive apodizations.

Each module in the ultrasonic imaging device may be implemented in whole or in part through software, hardware, or a combination thereof. The modules can be embedded in or independent of the processor in hardware form, or stored in the memory of the computer device in software form, so that the processor can call and execute the operations corresponding to each module. More descriptions regarding the modules may be found elsewhere in the present disclosure, for example, process 100 in FIG. 1.

In some embodiments, a computer device may be provided. In some embodiments, the computing device may be a terminal. FIG. 13 is a schematic diagram illustrating an exemplary computer device. As shown in FIG. 13, the computer device may include at least one processor, at least one memory, input/output interface, communication interface, display unit, and input apparatus. The processor, memory, and input/output interface may be connected via a system bus. The communication interface, display unit, and input device may be connected to the system bus through the input/output interface. The processor of the computer device may be used to provide computing and control capabilities. The storage of the computer device may include non-volatile storage media and/or internal memory. The non-volatile storage medium may store the operating system and/or computer programs. The internal memory may provide an environment for the operation of the operating system and computer programs in the non-volatile storage medium. The input/output interface of the computer device may be used for exchanging information between the processor and external devices. The communication interface of the computer device may be used for wired or wireless communication with the external terminals. Wireless communication can be achieved via WIFI, mobile cellular networks, Near Field Communication (NFC), or other technologies. The computer program may be executed by a processor to implement an ultrasonic imaging method. The display unit of the computer device may be used to form visually perceptible images. For example, the display unit may include a display screen, projection device, or virtual reality imaging device. The display screen may be a liquid crystal display or an e-ink display. The input device of the computer equipment may be a touch layer covering the display screen, buttons, a trackball, or a touchpad arranged on the computer device's casing, or an external keyboard, touchpad, or mouse.

As shown in FIG. 13, only a block diagram of part of the structure related to the solution of this application and does not constitute a limitation on the computer device to which the solution of this application is applied. Specific computer device may include more or fewer components than those shown in the FIG. 13, combine certain components, or have different component arrangements.

It should be noted that the processes 100, 1000 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes are intended to be illustrative. In some embodiments, the processes may be accomplished with one or more additional operations not described, and/or without one or more of the operations discussed. Additionally, the order in which the operations of the processes and regarding descriptions are not intended to be limiting.

In one embodiment, a computer device may be also provided, comprising a memory and a processor, wherein the memory stores a computer program, and the processor, when executing the computer program, implements the steps in the aforementioned ultrasonic imaging method.

It is to be noted that the above descriptions of the ultrasonic imaging system are provided for illustration purposes, and do not limit the present disclosure to the scope of the cited embodiments. It is to be understood that for a person skilled in the art, with an understanding of the principle of the system, it may be possible to arbitrarily combine the modules or form subsystems that are connected to other modules without departing from this principle.

In some embodiments, the obtainment module 310, the determination module 320, and the fusion module 330 disclosed in FIG. 12 may be different modules in a system, or one module realizing the functions of two or more other modules. For example, the modules may share a common storage module, or the modules may each have a respective storage module. Such deformations are within the scope of protection of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, FIG., or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. An ultrasonic imaging method, comprising:
for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets;
obtaining at least two initial ultrasonic images based on the at least two ultrasonic sub-images for the each of the at least one ultrasonic transmission; and
determining a target ultrasonic image of the scanning region by fusing the at least two initial ultrasonic images.

2. The ultrasonic imaging method according to claim 1, wherein the obtaining at least two ultrasonic sub-images of a scanning region acquired by at least two receiving array element subsets for each of at least one ultrasonic transmission comprises:
for the each of at least one ultrasonic transmission,
determining the at least two receiving array element subsets based on a preset selection rule;
acquiring ultrasonic echo signals through the at least two receiving array element subsets; and
obtaining the at least two ultrasonic sub-images based on the ultrasonic echo signals.

3. The ultrasonic imaging method according to claim 2, wherein at least two ultrasonic transmissions is performed and the obtaining the at least two initial ultrasonic images based on the ultrasonic sub-images comprises:
for each of the at least two ultrasonic transmissions, obtaining serial numbers of the at least two receiving array element subsets, and
obtaining the at least two initial ultrasonic images by coherently compounding at least two ultrasonic sub-images obtained based on ultrasonic echo signals collected by receiving array element subsets with the same serial number for the at least two ultrasonic transmissions.

4. The ultrasonic imaging method according to claim 3, wherein
the receiving array element subsets with the same serial number include different array element sets on an ultrasonic probe; or
the receiving array element subsets with the same serial number have different receiving apertures on the ultrasonic probe.

5. The ultrasonic imaging method according to claim 1, wherein the determining a target ultrasonic image of the scanning region by fusing the at least two initial ultrasonic images comprises:
obtaining at least two processed initial ultrasonic images by performing envelope detection on each of the at least two initial ultrasonic images; and
obtaining the target ultrasonic image of the scanning region by performing incoherent compounding on the at least two processed initial ultrasonic images.

6. The ultrasonic imaging method according to claim 1, wherein
for one of the at least one ultrasonic transmission,
each of the at least two receiving array element subsets includes different elements on an ultrasonic probe; or each of the at least two receiving array element subsets has different receiving apertures on the ultrasonic probe.

7. The ultrasonic imaging method according to claim 1, wherein
the at least two receiving array element subsets comprise a first receiving array element subset and a second receiving array element subset;
the first receiving array element subset and the second receiving array element subset include the same elements on an ultrasonic probe, or the first receiving array element subset and the second receiving array element subset have the same receive aperture on the ultrasonic probe; and
elements of the first receiving array element subset and elements of the second receiving array element subset have different receive apodization schemes.

8. The ultrasonic imaging method according to claim 1, wherein
at least two ultrasonic transmissions are performed;
a count of the at least two receiving array element subsets for each of the at least two ultrasonic transmissions is the same.

9. The ultrasonic imaging method according to claim 1, wherein
a count of the at least two receiving array element subsets is two; and
receiving apertures of the at least two receiving array element subsets are symmetrical relative to a transmission center of an ultrasonic probe.

10. The ultrasonic imaging method according to claim 1, wherein
a count of the at least two receiving array element subsets is two; and
receiving apertures of the at least two receiving array element subsets are asymmetrical relative to a transmission center of an ultrasonic probe.

11. The method of claim 1, wherein each of the at least two receiving array element subsets includes all elements on an ultrasonic probe or include a portion of the all elements on the ultrasonic probe.

12. An ultrasonic imaging system, comprising: a storage device, a processor, and instructions, being stored in the storage device, when executed by the processor, causing the ultrasonic imaging system to perform operations including:
for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets;
obtaining at least two initial ultrasonic images based on the at least two ultrasonic sub-images for the each of the at least one ultrasonic transmission; and
determining a target ultrasonic image of the scanning region by fusing the at least two initial ultrasonic images.

13. The ultrasonic imaging system of claim 12, further comprising a display, wherein the processor further causes the ultrasonic imaging system to perform operations including:
displaying the target ultrasonic image.

14. The ultrasonic imaging system according to claim 12, wherein at least two ultrasonic transmissions is performed and the obtaining the at least two initial ultrasonic images based on the ultrasonic sub-images comprises:
for each of the at least two ultrasonic transmissions, obtaining serial numbers of the at least two receiving array element subsets, and
obtaining the at least two initial ultrasonic images by coherently compounding at least two ultrasonic sub-images obtained based on ultrasonic echo signals collected by receiving array element subsets with the same serial number for the at least two ultrasonic transmissions.

15. The ultrasonic imaging system according to claim 14, wherein
the receiving array element subsets with the same serial number include different array element sets on an ultrasonic probe; or
the receiving array element subsets with the same serial number have different receiving apertures on the ultrasonic probe.

16. The ultrasonic imaging system according to claim 12, wherein the determining a target ultrasonic image of the scanning region by fusing the at least two initial ultrasonic images comprises:
obtaining at least two processed initial ultrasonic images by performing envelope detection on each of the at least two initial ultrasonic images; and
obtaining the target ultrasonic image of the scanning region by performing incoherent compounding on the at least two processed initial ultrasonic images.

17. The ultrasonic imaging system according to claim 12, wherein
for one of the at least one ultrasonic transmission,
each of the at least two receiving array element subsets includes different elements on an ultrasonic probe; or
each of the at least two receiving array element subsets has different receiving apertures on the ultrasonic probe.

18. The ultrasonic imaging system according to claim 12, wherein
the at least two receiving array element subsets comprise a first receiving array element subset and a second receiving array element subset;
the first receiving array element subset and the second receiving array element subset include the same elements on an ultrasonic probe, or the first receiving array element subset and the second receiving array element subset have the same receive aperture on the ultrasonic probe; and
elements of the first receiving array element subset and elements of the second receiving array element subset have different receive apodization schemes.

19. The ultrasonic imaging system according to claim 12, wherein
at least two ultrasonic transmissions are performed;
a count of the at least two receiving array element subsets for each of the at least two ultrasonic transmissions is the same.

20. A non-transitory computer readable medium, comprising at least one set of instructions, wherein when executed by one or more processors of a computing device, the at least one set of instructions causes the computing device to perform an ultrasonic imaging method comprising:
for each of at least one ultrasonic transmission, obtaining at least two ultrasonic sub-images of a scanning region acquired by at least two receiving array element subsets, each of the at least two ultrasonic sub-images being obtained based on ultrasonic echo signals collected by one of the at least two receiving array element subsets;
obtaining at least two initial ultrasonic images based on the at least two ultrasonic sub-images for the each of the at least one ultrasonic transmission; and
determining a target ultrasonic image of the scanning region by fusing the at least two initial ultrasonic images.
